(19) United States
(12) Patent Application Publication
(10) Pub. No.: US 2024/0206789 A1
(43) Pub. Date: Jun. 27, 2024

(54) System and Method for Measuring Wide Grip Upper Extremity Strength and Endurance

(71) Applicants: Paul Andrew Herman, Redondo Beach, CA (US); Mojca Cater Herman, Redondo Beach, CA (US)

(72) Inventors: Paul Andrew Herman, Redondo Beach, CA (US); Mojca Cater Herman, Redondo Beach, CA (US)

(21) Appl. No.: 18/089,460

(22) Filed: Dec. 27, 2022

(51) Int. Cl. A61B 5/22

(52) U.S. Cl. CPC A61B 5/225; A61B 2560/0462

(57) ABSTRACT

The invention relates to the creation of a device for the evaluation of sports specific wide grip strength and endurance for trainers and therapists. Devices that exist presently evaluate narrow grip and are not focused on athletes. The invention herein incorporates elements of sport to evaluate finger, thumb, and forearm strength. This invention provides a device that measures hand strength and endurance in a sports specific wide grip positions. In one embodiment, said device is comprised of a ball filled with a compressible gas or incompressible fluid with an orifice that allows pressure measurements to be taken. In another embodiment, the ball or an object is equipped with individual force sensors that will be aligned with the user's fingers and thumb. These configurations can be implemented independently or simultaneously. The data from the sensors will be made available to the athlete in real time.

### CROSS REFERENCE TO RELATED APPLICATIONS

This application claims the benefit of provisional patent application Ser. No. 63/307,148, filed Feb. 6, 2022.

### BACKGROUND—PRIOR ART

### U.S. Patents

### Patent Number Kind Code Issue Date Patentee

| | | | |
|---|---|---|---|
| 4,222,560 | A | 1980 Sep. 16 | Hallerman |
| 6,918,862 | B1 | 2005 Jul. 19 | Comeau |
| 8,607,869 | B2 | 2013 Dec. 10 | Miller |
| 9,439,594 | B2 | 2016 Sep. 13 | Akins |

### Non Patent Literature

Mathiowetz V., Weber K., Volland G. Kashman N. “Reliability and Validity of Grip and Pinch Strength Evaluations.” The Journal of Hand Surgery Volume 9, Issue 2, March 1984, Pages 222-226

Occupational and physical therapists commonly evaluate patient hand strength utilizing hydraulic and spring based grip and pinch gauges like the Jamar dynamometer and B&J pinch gauge. These instruments yield reliable and repeatable measurements that are used to evaluate the efficacy of therapeutic interventions being employed and have become the standard evaluation tool in hand therapy. However, these devices only evaluate the strength of the hand when in a “narrow” grip position. The hand is in a narrow grip position when gripping tools such as a hammer or holding a utensil such as a fork. However, when gripping a football or basketball, the hand must open up to accommodate the diameter of the ball prior to applying force. These standard therapy strength evaluation tools are inadequate for measuring hand strength when in a wide grip sports specific position.

Several alternatives have been proposed—for example, in U.S. Pat. No. 8,601,869 a multi-functional hand strength assessment device is proposed. This device utilizes a grip bar, a bellows, a pinch bar, and wheel shape that allows assessment of grip, pinch, and twisting strength in one device. The innovation of this device is the ability of one device to evaluate grip, pinch, and twisting with one device. The device is complicated and expensive to manufacture and does not evaluate sports specific wide grip strength.

U.S. Pat. No. 9,439,594 is a portable work capacity testing apparatus and method. This device is comprised of a portable computer, a hub, a strength and lifting device, a hand grip strength device, a pinch gauge, forearm/wrist strength device, a handling/proprioception device, a finger flexion device, a whole body coordination device, and a push/pull/lift device. The innovation of this device is its flexibility to measure many different aspects of strength of the human body. The main benefit is focused on the evaluation of workers and their ability to return to work in a manual labor setting. The device is complicated and expensive to manufacture and does not evaluate sports specific wide grip strength.

U.S. Pat. No. 6,918,862 is a hand strength exerciser and evaluator. This device incorporates both strengthening and measurement of strength into one unit. The device requires the user to squeeze a bladder to force the working fluid through a valve. A gauge is placed in the fluid path prior to the resistive valve, thereby measuring the strength of the squeeze, allowing the participant to track progress. This device is focused on grip strength in a different way than the commonly used clinical tools described above. However, it still focuses on the traditional closed/narrow grip and does not measure sports specific wide grip strength.

U.S. Pat. No. 4,222,560 proposes a variety of exercising devices achieved by varying the wall thickness and fluid viscosity thereby accommodating various hand strengths. A visual indicator of strength is provided by the amount of fluid ejected. This device focuses on the traditional closed/narrow grip and does not measure sports specific wide grip hand strength.

All of the proposed devices do not address sports specific wide grip evaluation or rehabilitation of hand strength. Athletes are a small niche of patients that require a different rehabilitation approach for upper extremity evaluation and rehabilitation. New and different tools are required to support athletes with upper extremity injuries. Their needs are different because they do different things with their hands, i.e. palming a basketball is very different than holding a fork or lifting a box. None of the prior art addresses the evaluation or rehabilitation of athletic wide grip strength. In addition, the utility of the devices proposed in the prior are limited by their complexity and resulting manufacturing expense.

This invention will now be described with respect to certain embodiments thereof as illustrated in the accompanying drawings, wherein:

### BRIEF DRAWING DESCRIPTIONS

FIG. 1 is a perspective view of the invention configured for hand grip using pressure measurements.

FIG. 2 is a perspective view of the invention configured solely for individual finger and or thumb measurement using a resistive/capacitive sensor.

FIG. 3 is an orthogonal view of the invention configured for hand grip using pressure measurements.

FIG. 4 is an orthogonal view of the invention configured solely for individual finger/thumb measurement using a resistive/capacitive sensor.

FIG. 5 is an enlarged orthogonal view of the force sensor platform shown in FIG. 4

FIG. 6 is a detailed perspective view of the sensing unit and display for the pneumatic/hydraulic sensor.

FIG. 7 is a detailed perspective view of the sensing unit and display for the resistive/capacitive sensor.

Flowchart 1 contains a logic description for the pneumatic/hydraulic system software.

Flowchart 2 contains a logic description for the resistive/capacitive force sensor system software.

### DESCRIPTION OF THE PREFERRED EMBODIMENTS

For purposes of the description hereinafter, spatial orientation terms, as used, shall relate to the referenced embodiment as it is oriented in the accompanying drawing figures or otherwise described in the following detailed description. However, it is to be understood that the embodiments described hereinafter may assume many alternative variations and configurations. It is also to be understood that the specific components, devices, and features illustrated in the accompanying drawing figures and described herein are simply exemplary and should not be considered limiting.

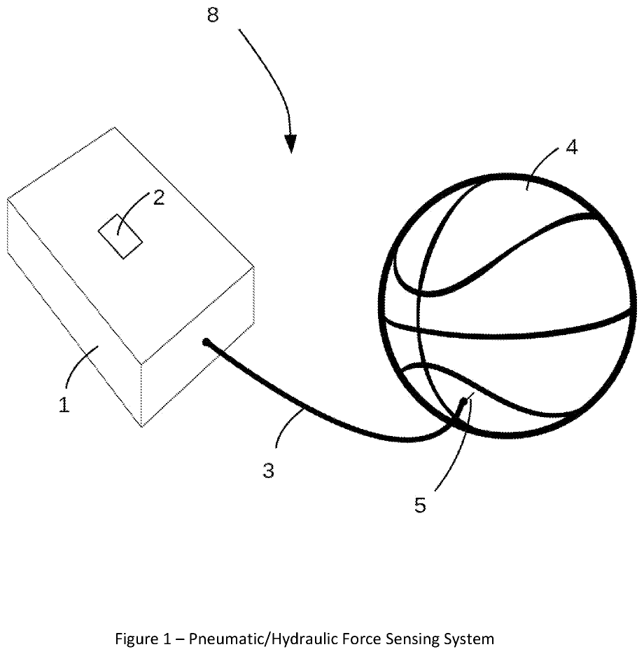

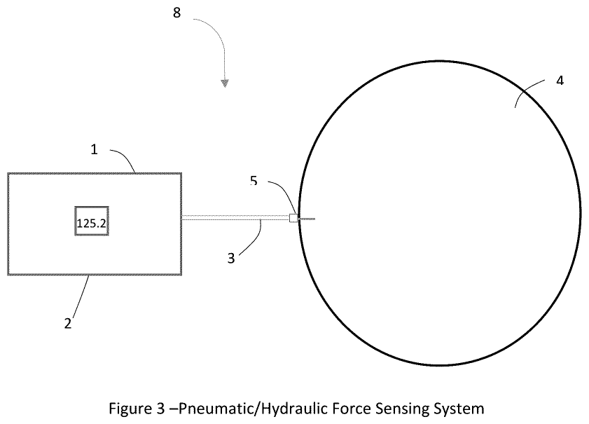

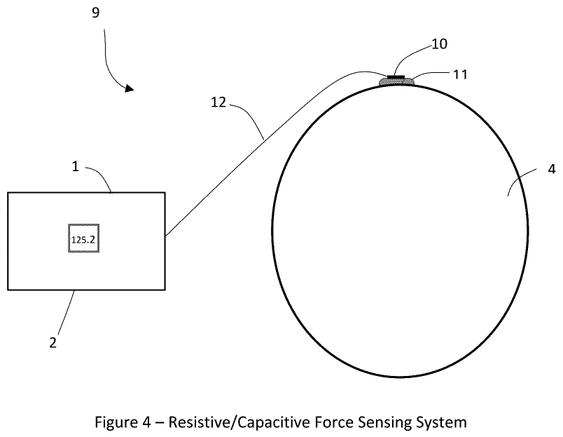

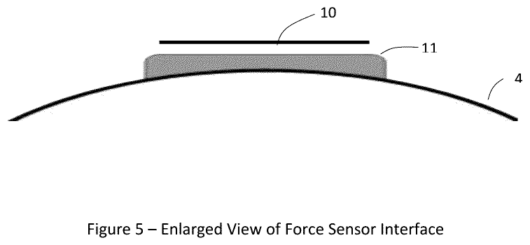

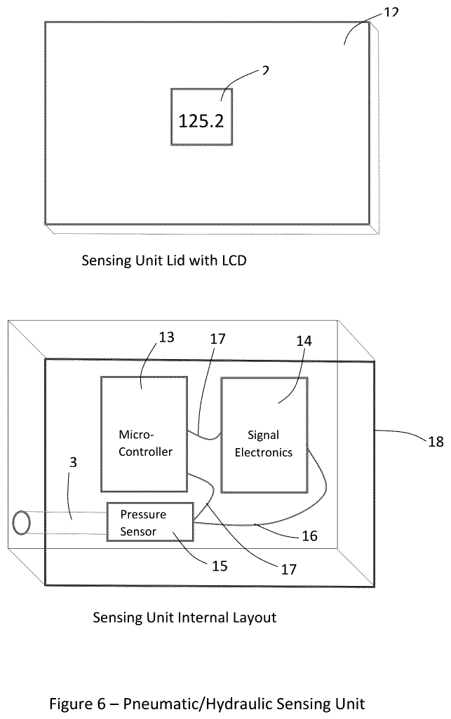

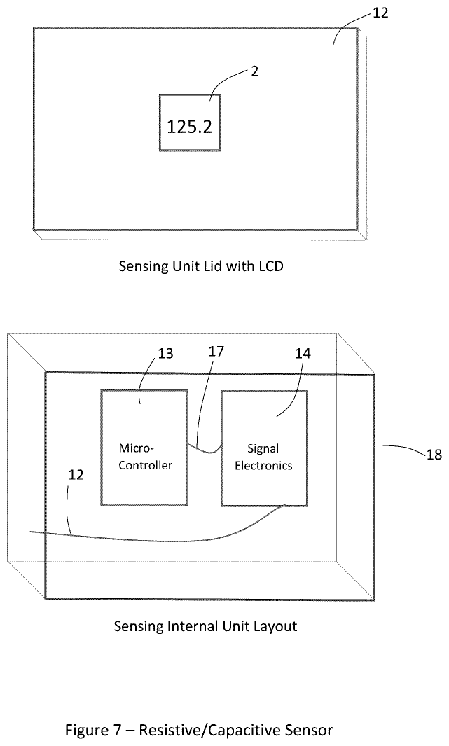

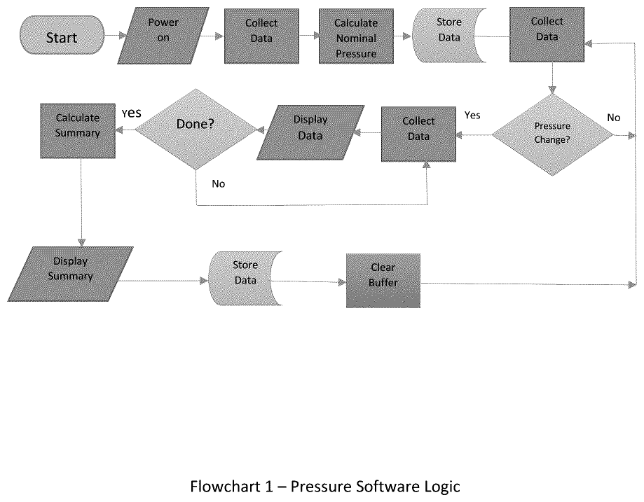

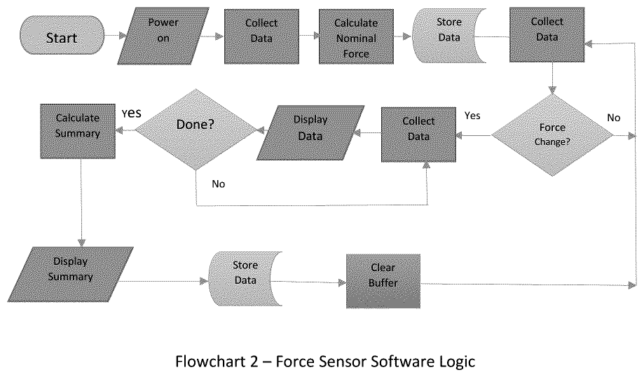

A sports specific wide grip upper extremity strength assessment device is shown in two different embodiments 8 and 9 in FIGS. 1-7. The invention permits quantification of sports specific wide grip upper extremity strength. Notably, wide sports specific upper extremity strength 8 and individual wide grip sport specific finger and thumb strength quantification is demonstrated 9.

The sports specific wide grip upper extremity strength assessment device 8, (hereinafter “device 8”) is comprised of a housing 1, LCD screen 2, gas/hydraulic tube 3, a ball 4, pressure probe 5, microcontroller 13, analog/digital electronics package 14, pressure sensor 15, electrical connections 16, 17, and 18. The housing 1 is comprised of a lid 12 and enclosure 18.

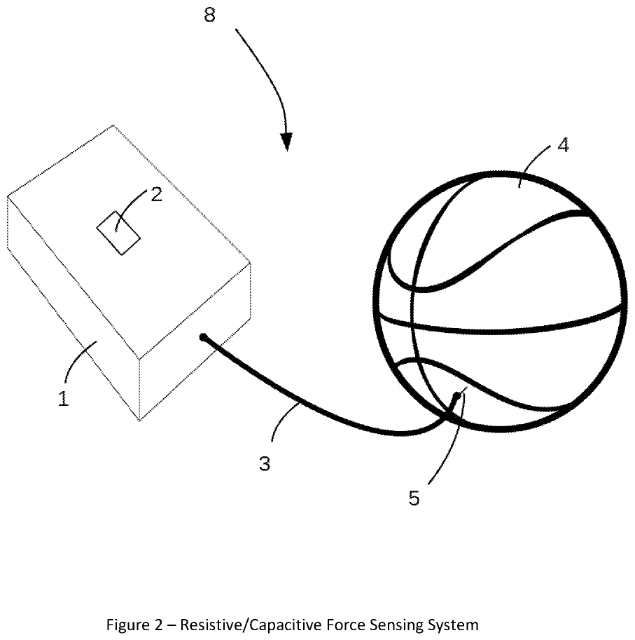

The sports specific wide grip individual finger and thumb strength assessment device 9, (hereinafter “device 9”) is comprised of a housing 1, LCD screen 2, a ball 4, wire harness 12, pressure sensor 10 and sensor platform 11, microcontroller 13, analog/digital electronics 14, and an electrical connection 17. It should be noted that the sub-assembly of pressure sensor 10 and platform 11 is labeled 7 in FIG. 2. The housing 1 is comprised of a lid 12 and enclosure 18.

The device 9 example provided in FIG. 2 displays only one sensor for simplicity. Although not shown, it is assumed that multiple sensors may be deployed simultaneously to evaluate multiple fingers and or thumb. It is also assumed that both device 8 and device 9 may be combined to take both sports specific upper extremity strength assessments with individual wide sports specific grip finger and thumb strength measurements simultaneously. It also assumed that a laptop or computer may be integrated into this design to augment or replace the sensing unit 1.

With continued reference to FIGS. 1-7, FLOWCHART 1, and FLOWCHART 2, operation of one embodiment device 8 to assess sports specific wide grip upper extremity strength consists of gripping the ball 4 creating increased pressure interior to ball. The pressure increase, due to squeezing the ball 4, can be better understood using the ideal gas law (1) which relates the relationship between pressure, volume, and temperature. Using equation (1), when the athlete grips the ball 4, the volume decreases resulting in an increase in internal pressure. This change is measured by the pressure sensor 15. This is expected because n, R, and T are assumed constant during the test period, therefore pressure is inversely proportional to the volume of the vessel.

PV=nRT (1)
P is pressure
V is volume
n is the amount of gas in moles (a constant)
R is the ideal gas constant
T is the absolute Temperature (Assumed constant for testing period)

The increased pressure is measured via the pressure probe 5, hydraulic tube 3 and pressure sensor 15. The microcontroller 13 and analog/digital electronics 14 process and filter the signal from the pressure sensor 15 to calculate the corresponding grip strength and display the value to the user via the LCD 2.

In another embodiment, device 9, the system does not rely on fluid pressure but instead on a simple inexpensive resistive or capacitive pressure sensor 10. When the athlete grips the ball, the sensor 10 is placed between the finger or thumb of interest and the ball 4. The sensor's resistance or capacitance changes due to the applied pressure. The change in resistance/capacitance is measured and filtered via the wire harness 12, analog/digital electronics 14, and micro controller 13. Feedback of the force being applied is displayed via LCD screen 2 for the user. The use of a sensor and platform combination 7 enables force measurements to occur on an unlimited number of form factors. It also allows data to be collected on individual fingers and thumbs. In this implementation, a key feature of the invention is the development of sensor/platform assembly 7 consisting of a platform 11 to mount the sensor 10. The platform 11 is curved to mount flush to the ball 4 but provides a flat area for sensor 10 mount to improve the accuracy and repeatability of measurements. Mounting 10 directly to the ball creates stress concentrations that can artificially inflate the force measurements and make them inconsistent from test to test.

In all the embodiments shown in FIGS. 1-7, the utilization of microcontroller 13, analog/digital electronics 14, and software (Flowchart 1 and Flowchart 2) allows for measurements such as average and peak upper extremity wide grip or average and peak individual thumb/digit strength. The software also allows for control of the duration or the test to evaluate endurance. These features are critical to assessing upper extremity wide grip strength and endurance. Another key feature of the software is the calibration performed during start up, noise rejection via multiple measurements, and automatic test start feature (Flowchart 1 and Flowchart 2) which allows test and retest without any additional inputs.

The invention has been described herein in terms of the preferred embodiments and methodologies considered by the inventors to represent the best mode of carrying out the invention. It will be understood by the skilled artisan, however, that a wide range of additions, deletions, and modifications, both subtle and gross, may be made to the illustrated and exemplary embodiments without departing from the spirit and scope of the invention. These and other revisions might be made by those with skill in the art without departing from the spirit and scope of the invention.

The invention claimed is:

1. A grip strength and endurance measuring device, comprising of a ball or other sports specific pressure vessels and pressure sensor, to measure wide grip strength and endurance including the forearm, hand, and thumb strength specifically for but not limited to the rehabilitation and strength improvement of athletes.

2. A grip strength and endurance measuring device, comprising of resistive and/or capacitive sensors mounted to a ball or other sports specific object to measure wide grip hand, thumb, digit, and forearm strength and endurance.

3. A sensor mounting platform consisting of a curved bottom with a flat top to integrate a resistive or capacitive pressure sensor on to a curved object to improve accuracy and repeatability of pressure sensor measurements.
